(19) United States
(12) Patent Application Publication
(10) Pub. No.: US 2007/0299318 A1
(43) Pub. Date: Dec. 27, 2007

(54) Medical monitoring device with remote transmission function

(75) Inventors: Kun-Sung Chen, Taipei (TW); Hsing Ou-Yang, Taipei (TW); Ying-Chao Lin, Taipei (TW)

Correspondence Address: BIRCH STEWART KOLASCH & BIRCH, PO BOX 747, FALLS CHURCH, VA, 22040-0747, US

(73) Assignee: Avita Corporation

(21) Appl. No.: 11/542,181

(22) Filed: Oct. 4, 2006

(30) Foreign Application Priority Data
Jun. 9, 2006 (TW) 95120512

(51) Int. Cl. A61B 5/00

(52) U.S. Cl. 600/300

(57) ABSTRACT

The present invention relates to a medical monitoring device with remote transmission function, comprising: a measuring unit; a storage unit; a mail server unit to provide mail sending/receiving service; a network server unit to provide Internet connection service; a web server unit to provide web-interfaced services; and an IP selection unit to provide a plurality of IP addresses connectable to the Internet and automatically set a different IP address for Internet connection periodically or each time the monitoring device is turned on, and send the set IP address to a designated e-mail box; wherein the stored medical data are made available for query via web interface.

### BACKGROUND OF THE INVENTION

1. Field of the Invention

The present invention relates to a remote healthcare system which uses a medical monitoring device with remote transmission function to send medical data measured to a remote user, thereby enabling the remote user to observe the physiological health condition of the patient being monitored.

2. Description of the Related Art

Elderly patients, newborn babies and patients with chronic illness often need long-term observation and care due to their feeble conditions. The healthcare personnel use all kinds of medical monitoring devices, e.g. blood pressure monitor, blood glucose meter, ear thermometer, body weight monitor, body fat analyzer and oximeter to measure the physiological indices of patients so as to keep track of their physical conditions.

Though the operation of those kinds of medical monitoring devices is made simpler by the advancement of technology, but any diagnosis based on the medical data measured requires the expertise of healthcare personnel. Thus some simple measuring tasks that could have been performed by the patient or his/her family at home often require the patient to go to the hospital or clinic so healthcare personnel can take measurement and observe. With the advent of medical equipment and the increasing popularity of home care, how to establish a remote healthcare system between the home of patient and the medical facility to allow healthcare personnel to carry out remote care is gaining more attention.

The establishment of a remote healthcares system allows patients who have difficulty moving around to check their physiological health conditions without the hassle of traveling back and forth a medical facility, and allow patients who require long-term monitoring and observation to have their physiological health conditions easily monitored at home without the hassle of constant trips between home and hospital. By having the patients or their family monitor their physical conditions at home, it saves the time of traveling and prevents the waste of medical human resources. The healthcare personnel can receive at a distant location the medical data of the home-cared patient and make professional judgment of the physical condition of the patient.

But the establishment of a medical healthcare system is faced with a vital issue. Currently such systems with remote transmission ability are web-based. In practice, the application of Internet has a data privacy problem. The health data of patients are personal private data that are protected by the privacy law. Thus how to protect the privacy of medical data transmitted over the web becomes a key point in the research and development of remote healthcare system.

### SUMMARY OF THE INVENTION

The object of the present invention is to provide a medical monitoring device with remote transmission function, which, through Internet connection, sends medical data measured by the medical monitoring device to a remote user to achieve the function of remote healthcare, and enhances the security of Internet transmission through a setting of multiple IP addresses.

The present invention provides a medical monitoring device with remote transmission function, which comprises: a monitoring unit to measure medical data; a storage unit to record the medical data measured; a mail server unit to provide mail sending/receiving service; a network server unit to provide Internet connection service; a web server unit to provide web-interfaced services; and an IP selection unit to provide a plurality of IP addresses connectable to the Internet and automatically set a different IP address for Internet connection on a periodic basis or when the medical monitoring device is turned on, and send the set IP address to a designated e-mail box; wherein stored medical data are made available for query via web interface.

According to the above invention, the medical monitoring device with remote transmission function according to the invention allows user at a remote distance, e.g. a medical facility or a relative of patient to observe the medical data of the patient measured at home through Internet. The device provided by the invention can communicate with other medical monitoring devices and carry out remote transmission of medical data measured by other monitoring devices. It also enhances the security of Internet transmission of medical data by automatically changing the IP address periodically or each time the device is turned on.

The object and features of the present invention are further illustrated with accompanying drawings, but the accompanying drawings and descriptions made in the examples should not be construed as a limitation or restriction on the actual application of the invention.

### BRIEF DESCRIPTION OF THE DRAWINGS

FIG. 1 is a block diagram of the medical monitoring device with remote transmission function according to the invention.

FIG. 2 is the structural diagram of an example of the device according to the invention in communication with other medical monitoring devices.

FIG. 3 is the flow chart of a blood pressure monitoring device according to an embodiment of the invention.

### DETAILED DESCRIPTION OF THE PREFERRED EMBODIMENTS

The present invention will be fully described with preferred embodiments and accompanying drawings. It should be understood beforehand that any person familiar with the skill is able to make modification to the invention described and attain the same effect, and that the description below is a general representation to people familiar with the skill and should not be construed as a limitation on the actual applicable scope of the present invention.

The medical monitoring device of the invention combines the measuring function of known medical monitoring instrument with data transmission function capable of remote communication to send the medical data measured to a remote user, thereby enabling the remote user, such as a healthcare personnel or relative of patient to monitor the physiological health condition of the patient.

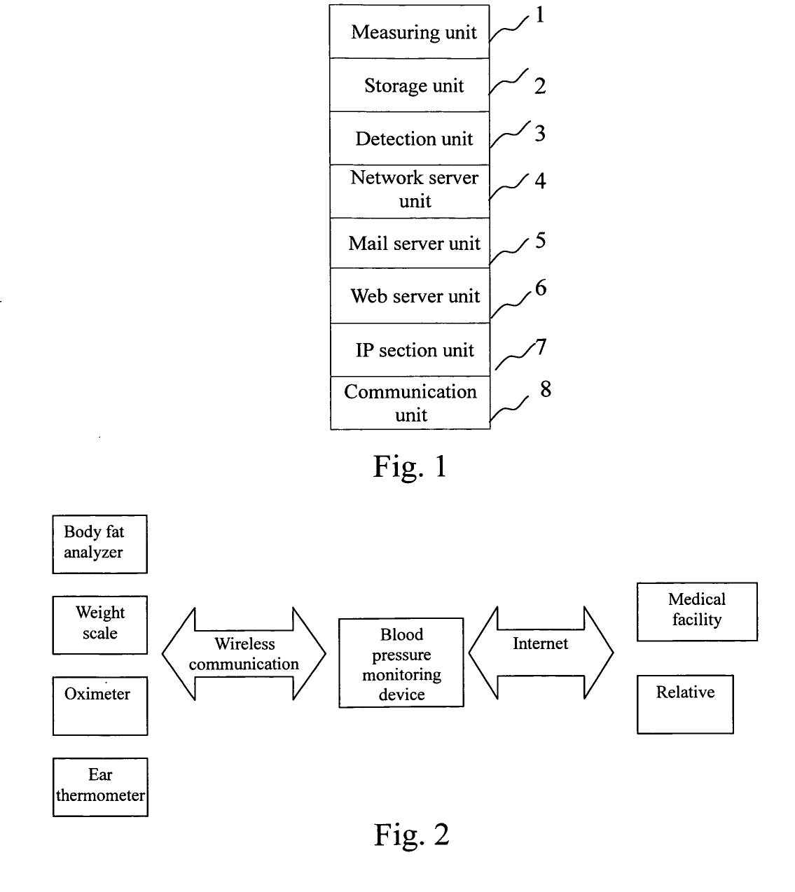

Referring to FIG. 1 which is a block diagram of the medical monitoring device with remote transmission function according to the invention, the device comprises a measuring unit 1 for measuring physiological health related data, e.g. blood pressure level, blood glucose level, blood oxygen level, blood lipid level, ear temperature, and body weight, and a storage unit 2 such as a hard disc or flash memory to store the medical data measured.

The device also comprises a network server unit 3 to provide Internet connection service so the device can perform remote data transmission via the Internet, and a mail server unit 4 to provide e-mail sending/receiving service that sends the medical data measured and stored or IP address of the device to a remote email box by electronic mailing. The device further comprises a web server unit 5 to provide web-interfaced services that allow remote users to connect to the device and make query of relevant medical data via web interface, or set designated e-mail address or available IP address for the medical monitoring device via web interface, or perform similar setting for other units.

The medical monitoring device also comprises a detection unit 3 to determine whether the measured medical data exceed a predefined normal range. When the measurement exceeds a predefined normal range, the detection unit 3 sends out an alarm message and uses the mail server unit 5 to send the alarm message to a designated mail address.

To enhance the confidentiality of data flow on the Internet, the medical monitoring device of the invention further comprises a IP selection unit 7, which provides a plurality of IP addresses connectable to the Internet to allow the device to change its IP address periodically or each time the device is turned on. As such, the data privacy is enhanced, making it more difficult for unauthorized third party to learn the IP address for Internet connection and preventing unauthorized access to the device and the medical data of patient.

To optimize the functions of the medical monitoring device, the device further comprises a communication unit 8 for communicating with other medical monitoring devices and receiving all kinds of medical data measured by other medical monitoring devices. The transmission of communication unit 8 between the device and other medical monitoring devices can be effected via the interface of WI-FI, bluetooth, infrared, RS232 or USB in a wired or wireless manner.

FIG. 2 shows the structural diagram of an example of the device according to the invention in communication with other medical monitoring devices. In this example, the monitoring device is a blood pressure monitoring device with a blood pressure measuring unit and using bluetooth or infrared to provide a wireless communication interface to communicate with other medical monitoring devices, e.g. body fat analyzer, weight scale, oximeter or ear thermometer. The blood pressure monitoring device can use the blood pressure measuring unit to measure the blood pressure of the patient, and receive and store data relating to the physiological health of the patient measured by other monitoring devices, e.g. blood oxygen level, body fat level, ear temperature and body weight.

The blood pressure monitoring device uses its detection unit to determine whether the stored medical data exceed a predefined normal range, and sends via web interface the medical data received to a remote user, such as a medical facility or relative of the patient by electronic mail, or provide the remote users with data query service via web interface.

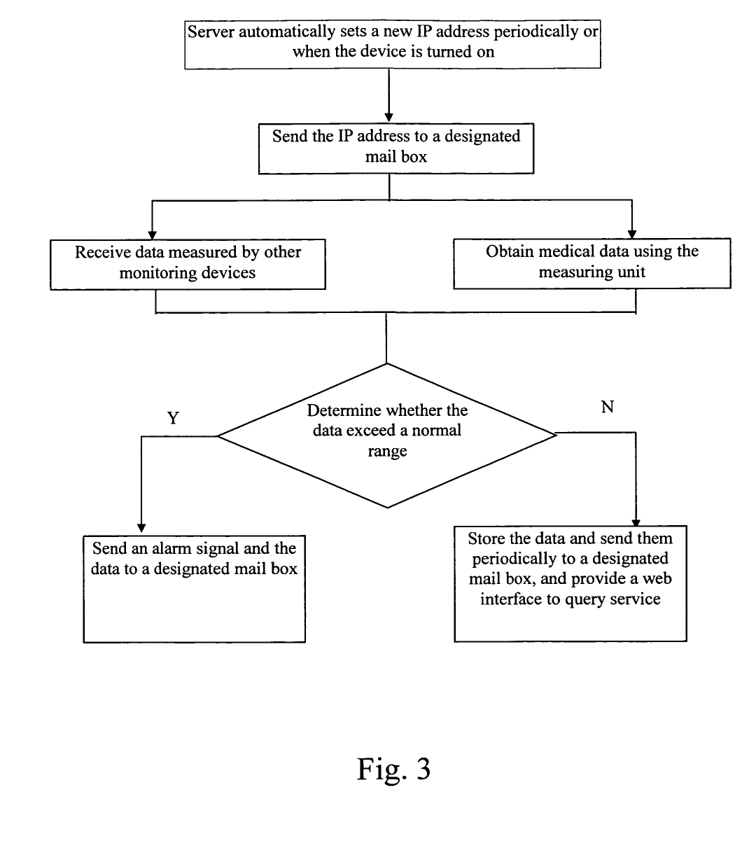

FIG. 3 is the flow chart of a blood pressure monitoring device according to an embodiment of the invention. The device automatically sets an IP address periodically or each time it is turned on, and sends the IP address to a designated mail box by electronic mail to let a remote user, such as a medical facility or relative of the patient to carry out remote connection using the IP address.

Subsequently the blood pressure monitoring device uses its blood pressure measuring unit to measure blood pressure and receives medical data measured by other monitoring devices, e.g. blood oxygen level, body fat level, ear temperature and body weight, and determines whether the data measured or received, e.g. blood pressure, blood oxygen, body fat, ear temperature or body weight exceed a predefined normal range using its detection unit. If the detected medical data exceed the predefined normal range, the blood pressure monitoring device would send an alarm signal and related medical data to a designated e-mail address by electronic mail to notify and allow the remote user, such as a medical facility or relative of the patient to respond swiftly to the abnormal condition. Conversely, if the medical data detected stay within the predefined normal range, the blood pressure monitoring device would store the blood pressure measured or medical data received from other monitoring units, e.g. blood oxygen level, body fat level, ear temperature and body weight in the storage unit, and send the stored data periodically to a designated e-mail address. The device also uses the web server unit to provide a web interface for remote users to make query of related medical data.

The medical monitoring device assembles the network server unit, mail server unit, and web server unit to achieve remote transmission capability via Internet connection, thereby allowing a medical facility or the relative of patient to check on the physiological health condition of the patient online. As such, patients do not need to visit a medical facility to get measurement of physiological data, while healthcare personnel can make diagnosis directly based on the medical data transmitted via the Internet. It saves patients from the toil of travel, and saves human resources, time and costs of healthcare.

The medical monitoring device of the invention can communicate with other medical monitoring devices with a wired or wireless communication unit that allows the medical data measured by other monitoring devices to be transferred remotely. As such, a remote user can use the medical monitoring device of the invention to monitor the medical data of patient measured by other monitoring devices. In consideration of the confidentiality of Internet data transfer, the IP selection unit of the medical monitoring device enables the device to be connected to the Internet via a different IP address periodically or each time the device is turned on. It prevents unauthorized access to the device and the medical data of patient through a stolen IP address.

The preferred embodiments of the present invention have been disclosed in the examples. All modifications and alterations by people skilled in the field without departing from the spirits of the invention shall remain within the protected scope and claims of the invention.

What is claimed is:

1. A medical monitoring device with remote transmission function, comprising:
a measuring unit for measurement of medical data;
a storage unit for recording said medical data measured;
a network server unit to provide Internet connection service;
a mail server unit to provide electronic mail sending and receiving service;
a web server unit to provide web-interfaced services; and
an IP selection unit to provide a plurality of IP addresses connectable to the Internet, and automatically set a different IP address for Internet connection on a periodic basis or each time said medical monitoring device is turned on, and send said set IP address to a designated electronic mail box;
wherein said medical monitoring devices stores said medical data measured and makes the stored data available for query via web interface.

2. The medical monitoring device as claimed in claim 1, wherein said measuring unit is a blood pressure measuring unit, a blood oxygen measuring unit, a blood lipid measuring unit, an ear temperature measuring unit, or a body weight measuring unit.

3. The medical monitoring device as claimed in claim 1, wherein a user can use the IP address received in said designated electronic mail box to connect to the web interface of said medical monitoring device to set said designated electronic mail box.

4. The medical monitoring device as claimed in claim 1, wherein further comprising a transmission interface to receive and store data measured by other medical monitoring devices.

5. The medical monitoring device as claimed in claim 4, wherein said transmission interface is a WI-FI, bluetooth, infrared, RS2323 or USB interface.

6. The medical monitoring device as claimed in claim 1, wherein further comprising a network camera to capture images and provide the service of image query via web interface.

7. The medical monitoring device as claimed in claim 1, wherein further comprising a detection unit to detect whether the medical data measured or received exceed a predefined value, when the medical data measured or received exceed a predefined value, said medical monitoring device will send an alarm signal to said designated electronic mail box.

8. A medical monitoring device with remote transmission function, comprising:
a measuring unit for measurement of medical data;
a storage unit for recording said medical data measured;
a network server unit to provide Internet connection service; and
a mail server unit to provide electronic mail sending and receiving service;
wherein said medical monitoring devices stores said medical data measured and sends the stored medical data by electronic mail to a designated electronic mail box.

9. The medical monitoring device as claimed in claim 8, wherein said measuring unit is a blood pressure measuring unit, a blood oxygen measuring unit, a blood lipid measuring unit, an ear temperature measuring unit, or a body weight measuring unit.

10. The medical monitoring device as claimed in claim 8, wherein further comprising a web server unit to provide web-interfaced services and the function of setting said designated electronic mail address.

11. The medical monitoring device as claimed in claim 8, wherein further comprising an IP selection unit to provide a plurality of IP addresses connectable to the Internet, and automatically set a different IP address for Internet connection on a periodic basis or each time said medical monitoring device is turned on, and send said set IP address to said designated electronic mail box.

12. The medical monitoring device as claimed in claim 8, further comprising a transmission interface to receive and store data measured by other medical monitoring devices.

13. The medical monitoring device as claimed in claim 12, wherein said transmission interface is a WI-FI, bluetooth, infrared, RS2323 or USB interface.

14. The medical monitoring device as claimed in claim 8, wherein further comprising a network camera to capture the images of person being monitored and provide the service of image query via web interface.

15. The medical monitoring device as claimed in claim 8, wherein further comprising a detection unit to detect whether the medical data measured or received exceed a predefined value; when the medical data measured or received exceed a predefined value, said medical monitoring device will send an alarm signal to said designated electronic mail box.
